(19) United States
(12) Patent Application Publication
(10) Pub. No.: US 2025/0180468 A1
(43) Pub. Date: Jun. 5, 2025

(54) Generating Parameters to Predict Hybridization Strength of Nucleic Acid Sequences

(71) Applicant: DNA Analytics, Greenbelt, MD (US)

(72) Inventors: Raymond J. Peterson, Greenbelt, MD (US); Jason D. Kahn, Chevy Chase, MD (US)

(21) Appl. No.: 18/843,332

(22) PCT Filed: Mar. 14, 2023
(86) PCT No.: PCT/US2023/064288

§ 371 Date: Sep. 3, 2024

Related U.S. Application Data
(60) Provisional application No. 63/269,294, filed on Mar. 14, 2022.

(51) Int. Cl. G01N 21/31; G16B 15/00

(52) U.S. Cl. CPC G01N 21/31; G16B 15/00; G01N 2021/3125

(57) ABSTRACT

A dilution of an oligonucleotide duplex is made. One aliquot of the dilution is added to a double-stranded nucleic acid (dsNA) melt at low concentration. A second aliquot of the dilution is added to a dsNA melt at high concentration. A third aliquot of the dilution is added to a reference cuvette containing only a first strand of the duplex. A fourth aliquot of the dilution is added to a reference cuvette containing only a second strand of the duplex. A first dsNA melt curve, a second dsNA melt curve, a first strand absorbance versus temperature curve, and a second strand absorbance versus temperature curve are produced. ΔH° and ΔS° are calculated for the first strand and the second strand from a fit to the first dsNA melt curve, the second dsNA melt curve, the first strand absorbance versus temperature curve, and the second strand absorbance versus temperature curve.

### INTRODUCTION

The teachings herein relate to methods that provide accurate predictions of the thermodynamic parameters for oligonucleotides containing more than one nucleic chemistry. More particularly the teachings herein relate to systems and methods for calculating the change in enthalpy and the change in entropy for the melting of individual oligonucleotides from experimental data.

The systems and methods herein can be performed in conjunction with a processor, controller, or computer system, such as the computer system of FIG. 1.

### BACKGROUND

Hybridization between complementary nucleic acids is an implicit feature in the Watson-Crick model for DNA structure that is exploited for many applications of the biological and biomedical arts. For example, virtually all methods for replicating and/or amplifying nucleic acid molecules are initiated by a step in which a complementary oligonucleotide (typically referred to as a “primer”) hybridizes to some portion of a “target nucleic acid molecule.” A polymerase then synthesizes a complementary nucleic acid from the primer, using the target nucleic acid as a “template.” See Kleppe et al., 1971. J. Mol. Biol. 56:341-61.

One application, known as the polymerase chain reaction, PCR, is widely used in a variety of biological and medical arts. For a description, see Saiki et al., 1985, Science 230; 1350-54. In PCR, two or more primers are used that hybridize to separate regions of a target nucleic acid and its complementary sequence. The sample is then subjected to multiple cycles of heating and cooling, repeatedly hybridizing, and dissociating the complementary Strands so that multiple replications of the target nucleic acid and its complement are performed. As a result, even very small initial quantities of a target nucleic acid can be enormously increased, or “amplified” for Subsequent uses (e.g., for detection, sequencing, etc.).

Multiplex PCR is a particular version of PCR in which several different primers are used to amplify and detect a plurality of different nucleic acids in a sample usually ten to a hundred or more different target nucleic acids. Thus, the technique allows a user to amplify and evaluate large numbers of different nucleic acids simultaneously in a single sample. The enormous benefits of high throughput, speed, and efficiency offered by this technique have made multiplex PCR increasingly popular. However, the achievement of successful multiplex PCR usually involves empirical testing as existing computer programs that pick and/or design PCR primers have errors. In multiplex PCR, the errors become additive, and therefore good results are seldom achieved without some amount of trial and error. See Markouatos et al., 2002, J. Clin. Lab Anal. 16 (1): 47-51; Henegarin et al., 1997, Biotechniques 23 (3): 504-11.

Some applications using probes and primers are designed to distinguish between two or more sequences that differ by one or more nucleotides, such as assays designed for single nucleotide polymorphism (SNP) detection. In these assays, mutations of clinical significance differ by a single nucleotide from the wild-type sequence.

Stability and melting temperature, Tm, of nucleic acid duplexes is a key design parameter for a variety of applications utilizing DNA and RNA oligonucleotides (Petersen and Wengel, 2003, Trends Biotechnol. 21:74-81: You et al., 2006, Nucleic Acids Res., 34: e60). The successful implementation of all techniques involving nucleic acid hybridization (including the exemplary techniques described, Supra) is dependent upon the use of nucleic acid probes and primers that specifically hybridize with complementary nucleic acids of interest while, at the same time, avoiding non-specific hybridization with other nucleic acid molecules that can be present. For a review, see Wetmur, 1991, Critical Reviews in Biochemistry and Molecular Biology 26:227-59. These properties are even more critical in techniques, such as multiplex PCR and microarray hybridization, where a plurality of different probes or primers is used, each of which can be specific for a different target nucleic acid.

Various modifications are available that can significantly affect the Tm of a nucleic acid duplex. The modifications can be placed at a terminal end, such as a minor groove binder (MGB) (Kutyavin et al., 2000, Nucleic Acids Research, 28 (2): 655-61). The modifications can be placed on the backbone of the oligonucleotide, examples of which include phosphorothioates, phosphorodithioates, and phosphonoacetates. The modifications can be located on the sugar moiety, examples of which include locked nucleic acids (LNAs), 2′-O-methyls, 2-methoxyethylriboses (MOEs), ENAs (ethylene bicyclic nucleic acids). The modification can be located on the base moiety, examples of which include 5-methyl-dC and propynyl-dU and propynyl-dC.

LNAs are RNA modifications wherein a methyl bridge connects the 2′-OXygen and the 4′-carbon, locking the ribose in an A-form conformation, providing synthetic oligonucleotides with unique properties (Koshkin et al., 1998, Tetrahedron 54; 3607-30; U.S. Pat. No. 6,268,490). LNA modifications increase the stability of nucleic acid duplexes and the specificity of oligonucleotide binding to complementary sequences, e.g., genomic DNAS (Petersen and Wengel, 2003). Therefore, oligonucleotides containing LNA modifications can be used to improve the accuracy and sensitivity of various biological applications and assays, e.g., antisense oligonucleotides, nucleic acid microarrays, sequencing, PCR primers, PCR probes, and medical diagnostics.

Preliminary work has been performed to develop thermodynamic parameters for DNA duplexes containing an LNA modification (see McTigue et al., 2004, Biochemistry 43 (18): 5388-05). McTigue et al. improved upon the older model of Tm, prediction simply based upon the number of LNA additions, and described sequence-dependent thermodynamic parameters for duplex formations containing a single LNA modification.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings can be implemented.

FIG. 2 is a schematic diagram of a system for estimating the change in enthalpy, ΔH°, and the change in entropy, ΔS°, for the melting of individual oligonucleotides from experimental data, in accordance with various embodiments.

FIG. 3 is an exemplary flowchart showing a method for estimating the change in enthalpy, ΔH°, and the change in entropy, ΔS°, for the melting of individual oligonucleotides from experimental data, in accordance with various embodiments.

FIG. 4 is a schematic diagram of a system that includes one or more distinct software modules and that performs a method for estimating the change in enthalpy, ΔH°, and the change in entropy, ΔS°, for the melting of individual oligonucleotides from experimental data, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

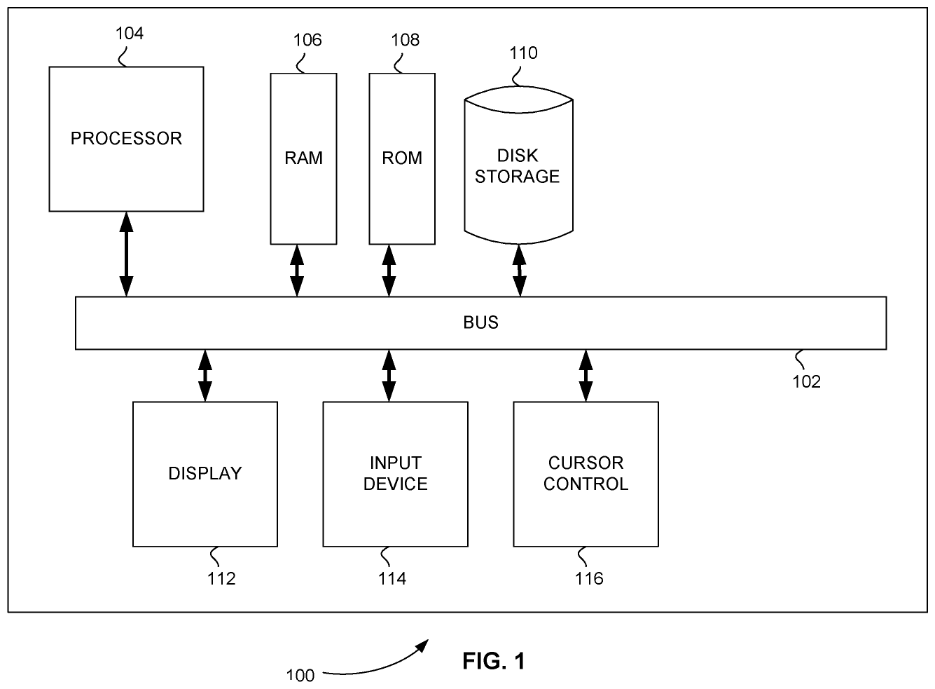

FIG. 1 is a block diagram that illustrates a computer system 100, upon which embodiments of the present teachings can be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also can be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 can be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions can be read into memory 106 from another computer-readable medium, such as storage device 110. Execution of the sequences of instructions contained in memory 106 causes processor 104 to perform the process described herein.

Alternatively, hard-wired circuitry can be used in place of or in combination with software instructions to implement the present teachings. For example, the present teachings may also be implemented with programmable artificial intelligence (AI) chips with only the encoder neural network programmed—to allow for performance and decreased cost. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product” are used interchangeably throughout this written description. Such a medium may take many forms, including but not limited to, non-volatile media and volatile media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media can be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or can be acquired from practicing of the present teachings. Additionally, the described implementation includes software but the present teachings can be implemented as a combination of hardware and software or in hardware alone. The present teachings can be implemented with both object-oriented and non-object-oriented programming systems.

### Generating Thermodynamic Parameters for Oligos

Embodiments of systems and methods for generating thermodynamic parameters for the melting of individual oligonucleotides from experimental data. In this detailed description, for purposes of explanation, numerous specific details are set forth to provide a thorough understanding of embodiments of the present invention. One skilled in the art will appreciate, however, that embodiments of the present invention may be practiced without these specific details. In other instances, structures and devices are shown in block diagram form. Furthermore, one skilled in the art can readily appreciate that the specific sequences in which methods are presented and performed are illustrative and it is contemplated that the sequences can be varied and remain within the spirit and scope of embodiments of the present invention.

In fitting Absorbance versus Temperature (“T”) curves, which are the raw data used for determining the thermodynamic parameters for melting of individual oligos, traditionally there are six parameters needed to fit each curve: the initial absorbance, the final absorbance, the slope of the absorbance versus T curve at low T, the slope at high T, and the desired concentration-independent parameters for enthalpy, ΔH°, and entropy, ΔS°. From ΔH° and ΔS°, the energy change, ΔG°, at any temperature and the melting temperature, Tm, at any concentration (CT) are readily calculated. There are two different commonly used ways to extract ΔH° and ΔS°. (A) One method is the direct fitting of the van′t Hoff equation to an individual melting curve. (B) The other method is measuring Tm as a function of concentration and fitting the equation the results to the second equation below. The two methods are equivalent, but the second method (B) essentially throws away all of the information from the shape of each individual melting curve.

In method (A), van′t Hoff: ln(K)=(−ΔH°/R)(1/T)+ΔS°/R, where ln K is determined from the experimental absorbance at each temperature.

In method (B), 1/T_{M}=R/ΔH° ln(CT)+ΔS°/ΔH, where the Tm is essentially the midpoint of the transition.

In various embodiments, several advances have been made in the treatment of the experimental data. First, all data are analyzed with equations that consider that the concentrations of the two strands of a duplex are not necessarily the same. Second, each strand concentration is measured as precisely as possible by making a single dilution of each oligo and then adding one aliquot to a double-stranded DNA melt at low concentration, a second aliquot to a double-stranded DNA (dsDNA) melt at high concentration, and a third aliquot to a reference cuvette containing only the single-stranded component (there are two of these). This provides two separate melt curves and the absorbance versus T for each single strand, which means that a baseline extrapolation at high temperature is not needed: the shape of the baseline is measured.

Third, the two separate dsDNA melt curves are analyzed with a single set of global parameters rather than fitting them separately and then averaging the answers. This allows the self-consistency of all the volume measurements to be checked and for pipetting errors to be corrected. In the end, two curves are fitted with a total of four concentration values (two for each of the two oligos in each melt), a single value for the hypochromicity of the dsDNA, a single value for the slope of the dsDNA absorbance versus T at low T per unit concentration, and ΔH° and ΔS°: this is a total of 8 parameters globally fit to four curves (2 dsDNA melts and 2 reference single-stranded DNAs (ssDNAs)), versus the traditional 12 parameters for two melts and then averaging ΔH° and ΔS°. In various embodiments, the method immediately identifies problematic melts that have concentrations that differ unacceptably from the expected values based on the dilutions used and optimizes to give self-consistent volumes that differ by a few percent from the target volumes. This reduces the error for ΔH° and ΔS° to less than about 8% while not requiring a wide range of concentrations. It could readily be extended to as many concentrations as desired, and the number of parameters would increase by only two concentrations per run rather than 6 parameters per run.

In various embodiments, another main advance is in the singular value decomposition analysis used to extract nearest-neighbor or other sequence-dependent basis set parameters from a set of ΔH° and ΔS° values for each dsDNA. Performing separate regressions for ΔG°37, ΔH°, and ΔS° often gives values that do not agree with the fundamental equation ΔG°=ΔH°−TΔS°, because the errors in the experimental numbers are highly correlated with each other. This is often dealt with by doing regressions for only ΔG° and ΔH° and then simply calculating ΔS° from the fundamental equation, but this approach throws away the independent error estimates for ΔS°.

Instead, in various embodiments, a single singular value decomposition (SVD) is performed that simultaneously optimizes the match to the experiment for ΔG°37, ΔH°, and ΔS° and minimizes deviations from the fundamental equation. This provides a parameter set for ΔG°37, ΔH°, and ΔS° that fits the experiment, has input from the independent error estimates for all three parameters, and enforces the fundamental equation. These accurate, precise, and self-consistent parameters can be used in downstream algorithms without worrying about whether they will give consistent results.

In various embodiments, training of algorithms that predict sequence-dependence hybridization strength of nucleic acid sequences is most efficient with a unified approach that includes sequence design, all model parameters (typically “nearest-neighbor” dinucleotides) are represented in a set of training sequences, sequences that are short enough to be informative but long enough to resemble those used in biotechnology applications, sequences have a constant core sequence that improves cost efficiency and allows comparisons among experiments.

Further, in various embodiments, an experimental method is provided that uses the direct measurement of each strand concentration to minimize experimental error in strand concentration. It is an analytical method for which measured single-strand concentrations and reference spectra are constraints used to estimate the true concentration in mixtures and thereby the fraction double strand of the duplex. Simultaneous global fitting to data obtained over two or more concentrations provides the four fundamental parameters (ΔH°, ΔS°, the temperature dependence of double-stranded DNA absorbance, and the double-stranded DNA hypochromicity) describing the melting of each duplex; ΔG°37 and the melting temperature Tm as a function of concentration are then readily derived.

As is typical, nearest-neighbor parameters used to make predictions about new sequences are derived via singular value decomposition of design matrices. This process has been streamlined and automated, and the analysis reweighted to emphasize higher-reliability data from melts. Also, the method has been improved to enforce adherence of parameter sets to the thermodynamic identity ΔG°37=ΔH°−TΔS°.

With regard to sequence design, one needs a set of duplexes and constituent single-strand sequences to have specific nucleotide sequence and chemical composition to comprehensively train each model parameter with at least one experiment and ideally two or more experiments. If there is just one experiment for a parameter, the parameter estimate is the direct measure and there is no estimate of variability. Two or more experiments per parameter, and ideally three or more, generate a more accurate estimate of the parameter value, generate an estimate of variation, and estimate precision. There need to be enough duplexes to obtain reasonably accurate and precise parameter estimates. Ideally, parameter representation in the set of sequences is balanced, whereby each parameter is represented about the same number of times, to obtain a consistent level of estimated error across parameters.

The experimental single-strand nucleotide sequences of the duplex are of at least 8 and no more than 14 nucleotides or nucleotide pairs, to be short enough to melt in a reasonable two-state manner, but long enough to more closely resemble the characteristics of duplexes of length used in life sciences applications, where length is the number of nucleotides or nucleotide pairs.

Experimental nucleotide sequences can have a defined region of nucleotide positions that vary in nucleotide composition and/or chemistry to efficiently train a desired model, and outside of this defined region, have a constant nucleotide sequence that acts as a core sequence to enable the re-use of components and decreased number of reference data sets and facilitate comparisons among experiments.

The typical experimental procedure is to obtain readings over temperature ramp separately for duplex and constituent single strands, where experimental readings of constituent single strands minimize experimental error due to inaccurate strand concentration at the time of parameter estimation. Most typically, experimental readings are optical, though fluorescent, electrochemical, or another detection method that yields an accurate measurement of duplex behavior is possible.

The analytical method uses a temperature-dependent absorbance curve of constituent single strands as a direct input, and simultaneous fitting to two or more data sets improves estimates of the true concentrations of each single strand in each mixture and thereby the fraction of double strand of the duplex. The method allows for the estimation and correction of small pipetting errors and the identification of anomalous data sets.

Simultaneous estimates of the two van′t Hoff equation parameters (ΔH° and ΔS°), the temperature dependence of double-stranded DNA absorbance, and the double-stranded DNA hypochromicity for a duplex are made by use of global fitting to readings obtained for the duplex at two or more duplex concentrations. ΔG°37 is derived from the fundamental relationship of ΔG°37=ΔH°−301.15*ΔS°, where 301.15 is the temperature in Kelvin corresponding to 37° Celsius. Tm is then readily calculated for any concentration.

ΔH° and ΔS° for nearest-neighbor dinucleotide steps are obtained from collected melt data using improved singular value decomposition methods. These methods enforce ΔG°37=ΔH°−301.15*ΔS° for parameters as well as the experiment, which provides more reliable and self-consistent predictions for new sequences.

The above procedure can be applied to any nucleic acid backbone chemistry or modified bases. Duplex nucleotides can be of uniform chemistry, where duplex nucleotides are natural DNA or natural RNA, where duplex nucleotides are a non-natural engineered chemistry, where duplex is a hybrid in which nucleotides of strand 1 are of uniform chemistry, nucleotides of strand 2 are of uniform chemistry, and chemistry of strand 1 differs from chemistry of strand 2. This hybrid includes where strand 1 nucleotides are natural DNA chemistry and strand 2 nucleotides are natural RNA chemistry, but other chemistry like 2′-O-Methyl is possible, and/or where strand 1 nucleotides are natural chemistry and strand 2 nucleotides are engineered chemistry, or where nucleotides on both strands are engineered chemistry. Nucleotides of strand 1 can be of uniform natural chemistry and nucleotides of strand 2 can be of mixed natural chemistry and engineered chemistry, where typically most nucleotides of strand 2 are of natural chemistry and just one or a few nucleotides are of the engineered chemistry. For example, the natural nucleotides of the uniform strand are DNA, the natural nucleotides of the composite strand are DNA, and the engineered chemistry of the composite strand is LNA.

The above procedure can be applied to nearest-neighbor model parameters for sequence context and/or chemistry for a specific nucleotide position in the duplex. For example, the chemistry of the nucleotide or duplex position of interest can be positioned at 5′ terminal, 5′ penultimate, internal and not terminal or penultimate, 3′ penultimate, or 3′ terminal.

The above procedure is equally applicable when all nucleotide pairs of the duplex have canonical base pairing, such as A:T, A:U, G:C and G:U, and when one or more nucleotide pairs of the duplex have non-canonical base pairing, such as A:G or G:T. One strand can have a 1 base overhang, known as dangling end, or both strands have a 1 base overhang at opposite ends of the duplex.

The above procedure can be applied to any combination of chemistry, position, and match. The sequence-dependence model can be dinucleotide (nearest-neighbor), trinucleotide (next-nearest-neighbor), or of arbitrary length (2, 3, 4, . . . ).

The energies of a measured model of a chemistry can be combined with the parameters of another chemistry to obtain a contrived model that is the best-case combination. Typically, an additive model is used. Consider the nominal dinucleotide 5′-AE-3′/3′-ZU-5′ that has not been measured but the nominal dinucleotides AE/UU and AZ/AU have been measured. The energy of the nominal dinucleotide AE/ZU can be contrived as the AA/UU foundation energy (delta) plus the effect (delta-delta) of AE/UU (AE/UU-AA/UU) plus the effect (delta-delta) AZ/AU (AZ/AU-AA/UU). This is possible for all combinations of chemistries and measured energies.

### System for Estimating ΔH° and ΔS°

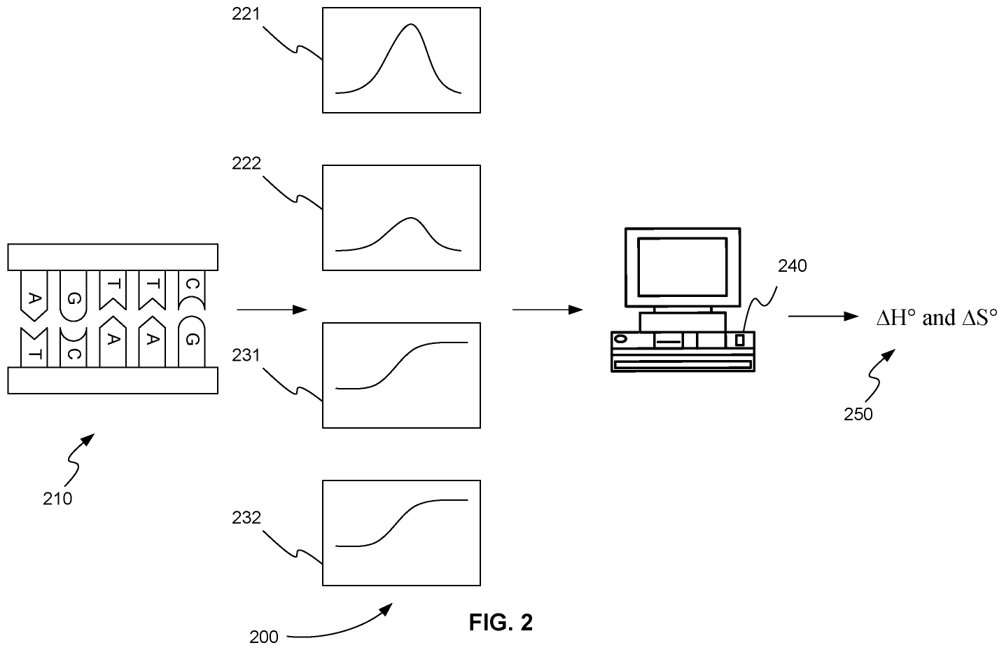

FIG. 2 is a schematic diagram 200 of a system for estimating the change in enthalpy, ΔH°, and the change in entropy, ΔS°, for the melting of individual oligonucleotides from experimental data, in accordance with various embodiments. The system includes processor 240. Processor 240 can be, but is not limited to, a controller, a computer, a microprocessor, the computer system of FIG. 1, or any device capable of analyzing data.

In step (a), Processor 240 receives a measured concentration of each strand of an oligonucleotide duplex 210. For example, a single dilution of duplex 210 is made. One aliquot of the dilution is added to a double-stranded nucleic acid (dsNA) melt at low concentration, producing a first dsNA melt curve 221. A second aliquot of the dilution is added to a dsNA melt at high concentration, producing a second dsNA melt curve 222. A third aliquot of the dilution is added to a reference cuvette containing only a first strand of duplex 210, producing a first strand absorbance versus temperature curve 231. Finally, a fourth aliquot of the dilution is added to a reference cuvette containing only a second strand of duplex 210, producing a second strand absorbance versus temperature curve 232.

In step (b), Processor 240 calculates ΔH° and ΔS° for the first strand from a fit to first dsNA melt curve 221, second dsNA melt curve 222, and first strand absorbance versus temperature curve 231. Processor 240 calculates ΔH° and ΔS° 250 for the second strand from a fit to first dsNA melt curve 221, second dsNA melt curve 222, and second strand absorbance versus temperature curve 232.

In various embodiments, processor 240 further calculates a first value for hypochromicity of the dsNA and a first value for a slope of the dsNA absorbance versus temperature at low temperature per unit concentration for the first strand from a fit to first dsNA melt curve 221, second dsNA melt curve 222, and first strand absorbance versus temperature curve 231. Processor 240 calculates a second value for hypochromicity of the dsNA and a second value for a slope of the dsNA absorbance versus temperature at low temperature per unit concentration for the second strand from a fit to first dsNA melt curve 221, second dsNA melt curve 222, and second strand absorbance versus temperature curve 232.

In various embodiments, oligonucleotide duplex 210 is a known oligonucleotide duplex. Processor 240 further performs steps (a) and (b) for one or more additional known oligonucleotide duplexes, producing a plurality of ΔH° and ΔS° values. Processor 240 then uses oligonucleotide duplex 210, the one or more additional known oligonucleotide duplexes, and their corresponding ΔH° and ΔS° values for each strand to create a mathematical model that can be used to predict the ΔH° and ΔS° values for an unknown oligonucleotide duplex. This mathematical model can be, for example, an artificial intelligence (AI) model or a machine learning model.

In various embodiments, steps (a) and (b) constitute a training algorithm that predict sequence-dependence hybridization strength of nucleic acid sequences in an efficient manner with a unified approach. This approach includes the following steps. Each strand concentration is directly measured to minimize experimental error in strand concentration. The measured single-strand concentrations and reference spectra are constraints used to estimate the true concentration in mixtures and thereby the fraction double strand of the duplex. Simultaneous global fitting is performed to data obtained over two or more concentrations to provide the four fundamental parameters (ΔH°, ΔS°, the temperature dependence of double-stranded DNA absorbance, and the double stranded DNA hypochromicity) describing the melting of each duplex; ΔG°37 and the melting temperature Tm as a function of concentration are then readily derived. Linear regression is applied by singular value decomposition of design matrices that enforces adherence of parameter sets to the thermodynamic identity ΔG°37=ΔH°−TΔS°.

In various embodiments, processor 240 further uses singular value decomposition analysis to extract nearest-neighbor or other sequence-dependent basis set parameters from a set of ΔH° and ΔS° values for each dsNA.

In various embodiments, duplex oligonucleotides are of uniform chemistry, natural DNA chemistry, or natural RNA chemistry.

In various embodiments, duplex oligonucleotides are of a non-natural engineered chemistry.

In various embodiments, duplex oligonucleotides are a hybrid in which nucleotides of a first strand are of uniform chemistry, nucleotides of second strand are of uniform chemistry, and the chemistry of the first strand differs from the chemistry of the second strand. In various embodiments, the first strand nucleotides are natural DNA chemistry and the second strand nucleotides are natural RNA chemistry. In various embodiments, the first strand nucleotides are natural chemistry and the second strand nucleotides are engineered chemistry.

In various embodiments, the nucleotides on both strands are engineered chemistry. In various embodiments, the nucleotides of the first strand are of uniform natural chemistry and nucleotides of the second strand are a composite of natural chemistry and engineered chemistry, where typically most nucleotides of the second strand are of natural chemistry and just one or a few nucleotides are of the engineered chemistry. In various embodiments, natural nucleotides of the uniform strand are DNA, natural nucleotides of the composite strand are DNA, and the engineered chemistry of the composite strand is LNA.

In various embodiments, model parameters for sequence context and/or chemistry are for a specific nucleotide position in the duplex, in which a specialized parameter set is provided for the nucleotide of interest being positioned at 5′ terminal position of the duplex; 5′ penultimate position; position is internal and not terminal or penultimate; 3′ penultimate; or 3′ terminal.

In various embodiments, the sequence-dependence model is dinucleotide (nearest-neighbor), trinucleotide (next-nearest-neighbor), or of arbitrary length (2, 3, 4, . . . ).

In various embodiments, the energies of a measured model of a chemistry may be combined with the parameters of another chemistry to obtain a contrived model that is the best-case combination, typically by use of an additive model.

In various embodiments, the system includes one ore more additional processors. The method is most useable to scientists when it yields predictions quickly and in a way that takes full advantage of compute resources. Towards this end, the algorithms are implemented with multiprocessing that makes optimal, concurrent use of all available compute CPUs, cores, and logical processors. When run concurrently, multiprocessing reduces task compute time up to 69%, as compared to single processing.

### Method for Estimating ΔH° and ΔS°

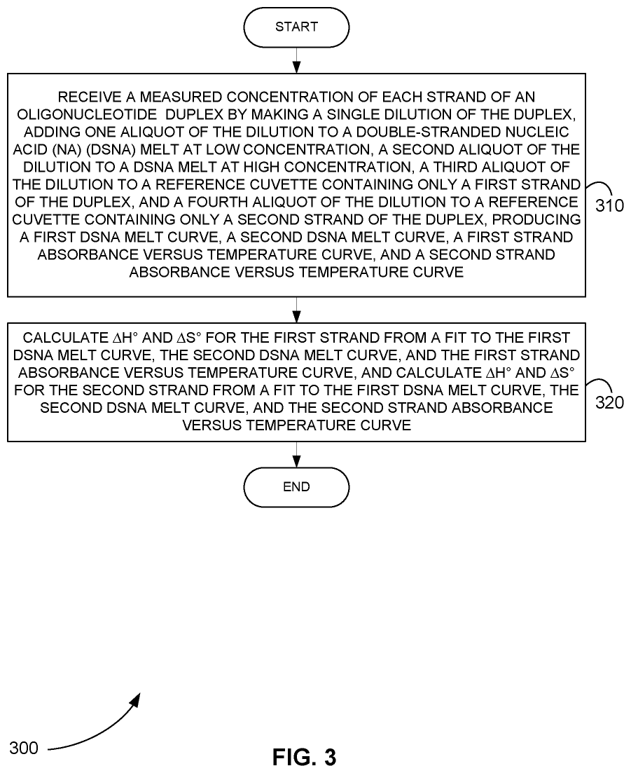

FIG. 3 is an exemplary flowchart showing a method 300 for estimating the change in enthalpy, ΔH°, and the change in entropy, ΔS°, for the melting of individual oligonucleotides from experimental data, in accordance with various embodiments.

In step 310 of method 300, a measured concentration of each strand of an oligonucleotide duplex is received. For example, a single dilution of the duplex is made. One aliquot of the dilution is added to a double-stranded nucleic acid (dsNA) melt at low concentration. A second aliquot of the dilution is added to a dsNA melt at high concentration. A third aliquot of the dilution is added to a reference cuvette containing only a first strand of the duplex. A fourth aliquot of the dilution is added to a reference cuvette containing only a second strand of the duplex. A first dsNA melt curve, a second dsNA melt curve, a first strand absorbance versus temperature curve, and a second strand absorbance versus temperature curve are produced.

In step 320, ΔH° and ΔS° are calculated for the first strand from a fit to the first dsNA melt curve, the second dsNA melt curve, and the first strand absorbance versus temperature curve. ΔH° and ΔS° are calculated for the second strand from a fit to the first dsNA melt curve, the second dsNA melt curve, and the second strand absorbance versus temperature curve.

### Computer Program Product for Estimating ΔH° and ΔS°

In various embodiments, a computer program product includes a non-transitory tangible computer-readable storage medium whose contents include a program with instructions being executed on a processor so as to perform a method for estimating the change in enthalpy, ΔH°, and the change in entropy, ΔS°, for the melting of individual oligonucleotides from experimental data. This method is performed by a system that includes one or more distinct software modules.

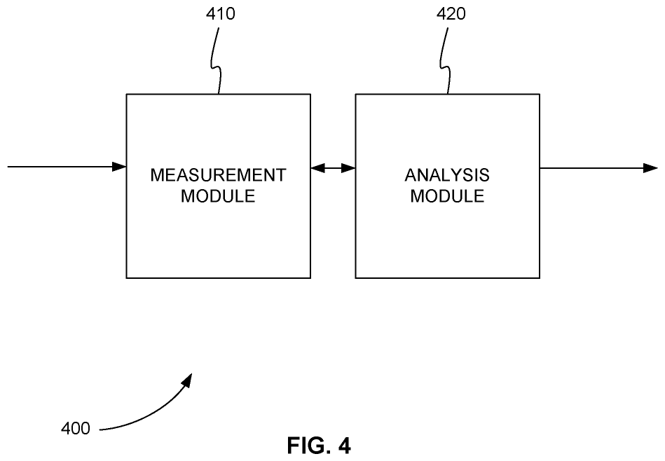

FIG. 4 is a schematic diagram of a system 400 that includes one or more distinct software modules and that performs a method for estimating the change in enthalpy, ΔH°, and the change in entropy, ΔS°, for the melting of individual oligonucleotides from experimental data, in accordance with various embodiments. System 400 includes measurement module 410 and analysis module 420.

Measurement module 410 receives a measured concentration of each strand of an oligonucleotide duplex. For example, a single dilution of the duplex is made. One aliquot of the dilution is added to a double-stranded nucleic acid (dsNA) melt at low concentration. A second aliquot of the dilution is added to a dsNA melt at high concentration. A third aliquot of the dilution is added to a reference cuvette containing only a first strand of the duplex. A fourth aliquot of the dilution is added to a reference cuvette containing only a second strand of the duplex. A first dsNA melt curve, a second dsNA melt curve, a first strand absorbance versus temperature curve, and a second strand absorbance versus temperature curve are produced.

Analysis module 420 calculates ΔH° and ΔS° for the first strand from a fit to the first dsNA melt curve, the second dsNA melt curve, and the first strand absorbance versus temperature curve. Analysis module 420 calculates ΔH° and ΔS° for the second strand from a fit to the first dsNA melt curve, the second dsNA melt curve, and the second strand absorbance versus temperature curve.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification can have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps can be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences can be varied and still remain within the spirit and scope of the various embodiments.

1. A method for estimating the change in enthalpy, ΔH°, and the change in entropy, ΔS°, for the melting of individual oligonucleotides from experimental data, comprising:
(a) receiving a measured concentration of each strand of a duplex by making a single dilution of the duplex, adding one aliquot of the dilution to a double-stranded nucleic acid (dsNA) melt at low concentration, a second aliquot of the dilution to a dsNA melt at high concentration, a third aliquot of the dilution to a reference cuvette containing only a first strand of the duplex, and a fourth aliquot of the dilution to a reference cuvette containing only a second strand of the duplex, producing a first dsNA melt curve, a second dsNA melt curve, a first strand absorbance versus temperature curve, and a second strand absorbance versus temperature curve; and
(b) calculating ΔH° and ΔS° for the first strand from a fit to the first dsNA melt curve, the second dsNA melt curve, and the first strand absorbance versus temperature curve, and calculating ΔH° and ΔS° for the second strand from a fit to the first dsNA melt curve, the second dsNA melt curve, and the second strand absorbance versus temperature curve.

2. The method of claim 1, further comprising calculating a first value for hypochromicity of the dsNA and a first value for a slope of the dsNA absorbance versus temperature at low temperature per unit concentration for the first strand from a fit to the first dsNA melt curve, the second dsNA melt curve, and the first strand absorbance versus temperature curve, and calculating a second value for hypochromicity of the dsNA and a second value for a slope of the dsNA absorbance versus temperature at low temperature per unit concentration for the second strand from a fit to the first dsNA melt curve, the second dsNA melt curve, and the second strand absorbance versus temperature curve.

3. The method of claim 1, further comprising using singular value decomposition analysis to extract nearest-neighbor or other sequence-dependent basis set parameters from a set of ΔH° and ΔS° values for each dsNA.

4-15. (canceled)
